(19) United States
(12) Patent Application Publication
(10) Pub. No.: US 2025/0017986 A1
(43) Pub. Date: Jan. 16, 2025

(54) SPRAY-DRIED COMPOSITIONS AND METHODS OF PREPARATION

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Sie Huey LEE, Singapore (SG); Wen Chien Desmond HENG, Singapore (SG); Siak Wei OW, Singapore (SG); Shao Quan LIU, Singapore (SG); Pui Shan CHOW, Singapore (SG); Pei Kun Richie TAY, Singapore (SG); Dai CHEN, Singapore (SG)

(21) Appl. No.: 18/705,455

(22) PCT Filed: Oct. 26, 2022
(86) PCT No.: PCT/SG2022/050763

§ 371 Date: Apr. 26, 2024

(30) Foreign Application Priority Data
Oct. 27, 2021 (SG) 10202111955W

(51) Int. Cl. A61K 35/747; A23C 9/16; A23G 9/36; A61K 9/50; A61K 35/00; A61K 35/741; A61K 35/742; A61K 35/744; A61K 35/745; C12C 5/00

(52) U.S. Cl. CPC A61K 35/747; A23C 9/16; A23G 9/363; A61K 9/5026; A61K 9/5089; A61K 35/741; A61K 35/742; A61K 35/744; A61K 35/745; C12C 5/00; A23V 2400/175; A61K 2035/115

(57) ABSTRACT

Spray-dried Compositions and Methods of Preparation Spray-dried compositions that may be used in food products, the methods of preparing the compositions and food products containing the compositions are described. The spray-dried composition contains 5 to 50 weight percent (wt. %) of a prebiotic. 10 to 65 wt. % of a probiotic, and 30 to 80 wt. % of a coating material. The prebiotic may be a polysaccharide, an oligosaccharide, a polyol, whey protein, and any combinations thereof. The probiotic may be Lacticaseibacillus, Lactobacillus, Lactiplantibacillus, Levilactobacillus, Ligilactobacillus, Limosilactobacillus, Bifidobacterium, Enterococcus, Streptococcus, Pediococcus, Leuconostoc, Bacillus, Escherichia, Saccharomyces, and any combinations thereof. The coating material may be a synthetic polymer, a natural polymer, and any combinations thereof. The spray-dried compositions are prepared by providing at least one solution containing the composition components and spray drying the at least one solution.

### TECHNICAL FIELD

The present invention relates to spray-dried compositions to improve the delivery of probiotics in food, and methods of preparing the compositions.

### BACKGROUND

Probiotics are live microbes, typically from the Lactobacillus, Bifidobacterium, or Bacillus genera, that confer beneficial health effects when consumed in adequate amounts [1]. At present, several international bodies have stated that a food product must contain at least 10 million live bacteria per gram of food to exert a functional effect within the body [1, 2, 3]. However, these probiotic strains often fall short of the viability requirements in final food products and upon passage through the gastrointestinal tract [1]. This is due to the harsh conditions (e.g. high temperature, pH/acidity, salinity) encountered by the probiotic bacteria during food processing, distribution/storage and digestion, which threatens their survival and functionality [4]. Currently, there is an emerging trend to incorporate probiotics into foods, especially non-dairy foods. Drying is often utilized to maximize the shelf-life of the probiotics (stable cold chain-free solid forms), however, the process inadvertently induces stress on the organisms, hence decreasing their viability [5]. Dried probiotics may not be in an active state that is more effective. Therefore, the identification and development of new protection strategies (e.g. benign drying technologies, use of protective excipients, novel microencapsulation strategies) is needed [5, 6].

### SUMMARY

In a first aspect, there is provided a spray-dried composition comprising a prebiotic, a probiotic, and a coating material.

In an embodiment, the spray-dried composition comprises 5 to 50 weight percent (wt. %) of a prebiotic, 10 to 80 wt. % of a probiotic, and 15 to 80 wt. % of a coating material. In an embodiment, the spray-dried composition comprises 5 to 50 weight percent (wt. %) of a prebiotic, 10 to 65 wt. % of a probiotic, and 30 to 80 wt. % of a coating material, preferably the prebiotic is selected from the group consisting of a polysaccharide, an oligosaccharide, a polyol, whey protein, and any combinations thereof; the probiotic is from a genus selected from the group consisting of Lacticaseibacillus, Lactobacillus, Lactiplantibacillus, Levilactobacillus, Ligilactobacillus, Limosilactobacillus, Bifidobacterium, Enterococcus, Streptococcus, Pediococcus, Leuconostoc, Bacillus, Escherichia, Saccharomyces, and any combinations thereof; and the coating material is selected from the group consisting of a synthetic polymer, a natural polymer, and any combinations thereof. Advantageously, the use of a naturally occurring polymer may be safer for human use. Examples of natural polymers that may be used as the coating material include polyols and proteins. It will be appreciated that naturally occurring polymers may be synthesised by chemical and/or biochemical methods. The coating material should be able to provide a good and efficient enteric coating in the gastric environment which food passes through after consumption.

Preferably, the probiotic is present in 10 to 60 wt. %, preferably 10 to 50 wt. %, more preferably 10 to 40 wt. %, and even more preferably 10 to 30 wt. %.

Preferably, the prebiotic is selected from the group consisting of soluble soy polysaccharides, maltodextrin, inulin, oligofructose, fructo-oligosaccharides, galacto-oligosaccharides, pectic oligosaccharides, xylooligosaccharides, soya bean oligosaccharides, isomaltooligosaccharides, glucooligosaccharides, arabinoxyloligosaccharides, raffinose, palatinose, lactosucrose, resistant starch and polydextrose, plant-derived and fungal-derived polysaccharides, mannitol, xylitol, sorbitol, lactulose, lactitol, maltitol, trehalose, lactose, whey protein isolate, whey protein concentrate, and any combinations thereof. More preferably, the prebiotic is the polysaccharide, even more preferably the prebiotic comprises soluble soy polysaccharides and/or maltodextrin.

In an embodiment, the genus of the probiotic is from a Lacticaseibacillus genus or a Lactobacillus genus. Preferably, the probiotic is Lacticaseibacillus, in particular the species is Lacticaseibacillus rhamnosus. In an embodiment, the genus of the probiotic is Escherichia, preferably the species is Escherichia coli.

Preferably, the coating material is selected from the group consisting of a polymer of acrylic acid and/or acrylates, shellac, alginic acid and its salts, pullulan, polyvinyl alcohol, polyvinylpyrrolidone, polylactic acid, polylactic-co-glycolic acid, polyethylene glycol, zein, chitosan, lipids, beewax, gums, cellulose and its derivatives, dextran, gelatine, bacterial cell wall, trehalose, mannitol, lactose, sucrose, erythritol, whey, soy protein, and any combinations thereof.

Preferably, the synthetic polymer and/or the natural polymer of the coating material each have an acid or ester functional group.

In an embodiment, the polymer of acrylic acid and/or acrylates is selected from the group consisting of poly(ethylacrylate-co-methyl methacrylate), poly(methacrylic acid-co-methyl methacrylate), poly(methacrylic acid-co-ethyl acrylate), poly(methyl acrylate-co-methyl methacrylate-co-methacrylic acid), and any combinations thereof. Preferably, the polymer of acrylic acid and/or acrylates is selected from the group consisting of poly(ethylacrylate-co-methyl methacrylate) with an approximate ratio of 2:1 to 1:1 of ethyl acrylate to methyl methacrylate, poly(methacrylic acid-co-methyl methacrylate) with an approximate ratio of 1:1 to 1:2 of methacrylic acid to methyl methacrylate, poly(methacrylic acid-co-ethyl acrylate) with an approximate ratio of 1:1 of methacrylic acid to ethyl acrylate, poly(methyl acrylate-co-methyl methacrylate-co-methacrylic acid) with an approximate ratio of 7:3:1 of methyl acrylate to methyl methacrylate to methacrylic acid, and any combinations thereof. Advantageously, copolymers of acrylic acid and acrylates are effective enteric coating materials.

Preferably, there are at least two different polymers. Advantageously, this allows the different polymers to each provide their benefit to the coating material and allows for more formulations to be developed for different food matrices.

Preferably, the prebiotic is distinct or different from the coating material.

In an embodiment, the spray-dried composition comprises 5 to 50 wt. % of a prebiotic, wherein the prebiotic is a polysaccharide, 10 to 80 wt. % of a probiotic, in which the genus of the probiotic is a Lacticaseibacillus genus or a Lactobacillus genus, and 15 to 80 wt. % of a coating material in which the synthetic polymer and/or the natural polymer of the coating material each have an acid or ester functional group. Preferably, at least one of the following conditions are fulfilled: (i) the polysaccharide is soluble soy polysaccharides and/or maltodextrin, (ii) the probiotic is a Lacticaseibacillus genus, and (iii) the coating material is selected from the group consisting of a polymer of acrylic acid and/or acrylates, shellac, and alginic acid and its salts.

Preferably, the spray-dried composition has one of the following compositions: (i) Lacticaseibacillus rhamnosus GG is present in 15 to 22 wt. %, soluble soy polysaccharides are present in 9-11 wt. %, sodium alginate is present in 33-35 wt. %, and shellac is present in 36-39 wt./c; (ii) Lacticaseibacillus rhamnosus GG is present in 15 to 22 wt. %, soluble soy polysaccharides are present in 33-35 wt. %, poly(ethylacrylate-co-methyl methacrylate) 2:1 is present in 18-20 wt. %, and shellac is present in 27-30 wt. %; and (iii) Lacticaseibacillus rhamnosus GG is present in 15 to 22 wt. %, soluble soy polysaccharides are present in 9-11 wt. %, maltodextrin is present in 33-35 wt. %, poly(ethylacrylate-co-methyl methacrylate) 2:1 is present in 9-10 wt. %, and shellac is present in 27-29 wt. %.

Preferably, at least one of the following conditions are fulfilled: (i) a majority of the particles in the spray-dried composition has a rough surface; (ii) the spray-dried composition is prepared by a two-fluid nozzle; (iii) a majority of the particles in the spray-dried composition has a smooth and dimpled surface; (iv) the spray-dried composition is prepared by a three-fluid nozzle.

Preferably, the spray-dried composition has a water activity of less than 0.6, more preferably 0.55 or less.

In a second aspect, there is provided a method of preparing a spray-dried composition, the method comprising: providing at least one solution comprising at least one prebiotic, at least one probiotic, and at least one coating material; and spray drying the at least one solution to obtain the spray-dried composition.

In an embodiment, the at least one solution comprises 5 to 50 weight percent (wt. %) of a prebiotic, 10 to 80 wt. % of a probiotic, and 15 to 80 wt. % of a coating material.

In an embodiment, the at least one solution comprises 5 to 50 weight percent (wt. %) of a prebiotic, 10 to 65 wt. % of a probiotic, and 30 to 80 wt. % of a coating material, wherein the prebiotic is selected from the group consisting of oligosaccharides, polysaccharides, polyols, whey protein and any combinations thereof, the probiotic is selected from the group consisting of Lacticaseibacillus, Lactobacillus, Lactiplantibacillus, Levilactobacillus, Ligilactobacillus, Limosilactobacillus, Bifidobacterium, Enterococcus, Streptococcus, Pediococcus, Leuconostoc, Bacillus, Escherichia coli, Saccharomyces, and any combinations thereof; the coating material is selected from the group consisting of a synthetic polymer, a natural polymer, and any combinations thereof.

Preferably, the probiotic is present in 10 to 60 wt. %, preferably 10 to 50 wt. %, more preferably 10 to 40 wt. %, and even more preferably 10 to 30 wt. %.

Preferably, at least one of the following conditions is fulfilled: (a) the prebiotic is selected from the group consisting of soluble soy polysaccharides, maltodextrin, inulin, oligofructose, fructo-oligosaccharides, galacto-oligosaccharides, pectic oligosaccharides, xylooligosaccharides, soya bean oligosaccharides, isomaltooligosaccharides, glucooligosaccharides, arabinoxyloligosaccharides, raffinose, palatinose, lactosucrose, resistant starch, polydextrose, plant-derived and fungal-derived polysaccharides, mannitol, xylitol, sorbitol, lactulose, lactitol, maltitol, trehalose, lactose, whey protein isolate, whey protein concentrate, and any combinations thereof; (b) the prebiotic is the polysaccharide, preferably soluble soy polysaccharides and/or maltodextrin; (c) the genus of the probiotic is Lacticaseibacillus or Lactobacillus, preferably the species is Lacticaseibacillus rhamnosus, more preferably Lacticaseibacillus rhamnosus GG; (d) the coating material is selected from the group consisting of a polymer of acrylic acid and/or acrylates, shellac, alginic acid and its salts, pullulan, polyvinyl alcohol, polyvinylpyrrolidone, polylactic acid, polylactic-co-glycolic acid, polyethylene glycol, zein, chitosan, lipids, beewax, gums, cellulose and its derivatives, dextran, gelatine, bacterial cell wall, trehalose, mannitol, lactose, sucrose, erythritol, whey, soy protein, and any combinations thereof; (c) the synthetic polymer and/or the natural polymer of the coating material each have a carboxylic acid functional group or an ester functional group; (f) there are at least two different polymers; and (g) the prebiotic is distinct or different from the coating material.

Preferably, the polymer of acrylic acid and/or acrylates is selected from the group consisting of poly(ethylacrylate-co-methyl methacrylate), poly(methacrylic acid-co-methyl methacrylate), poly(methacrylic acid-co-ethyl acrylate), poly(methyl acrylate-co-methyl methacrylate-co-methacrylic acid), and any combinations thereof. More preferably, the polymer of acrylic acid and/or acrylates is selected from the group consisting of poly(ethylacrylate-co-methyl methacrylate) with an approximate ratio of 2:1 to 1:1 of ethyl acrylate to methyl methacrylate, poly(methacrylic acid-co-methyl methacrylate) with an approximate ratio of 1:1 to 1:2 of methacrylic acid to methyl methacrylate, poly(methacrylic acid-co-ethyl acrylate) with an approximate ratio of 1:1 of methacrylic acid to ethyl acrylate, poly(methyl acrylate-co-methyl methacrylate-co-methacrylic acid) with an approximate ratio of 7:3:1 of methyl acrylate to methyl methacrylate to methacrylic acid, and any combinations thereof.

In an embodiment, spray drying the at least one solution is performed with a two fluid nozzle, preferably, the at least one solution comprises an aqueous solution.

In an embodiment, spray drying the at least one solution is performed with a three fluid nozzle, preferably the at least one solution comprises an aqueous solution and a solution with an organic solvent.

In a third aspect, there is provided a food product comprising the spray-dried composition according to the first aspect or prepared by the second aspect.

Advantageously, with a three fluid nozzle an aqueous solution and a solution with an organic solvent (may also be termed an organic solution) may be used, and allows a broader choice of excipients or components including water-insoluble excipients that are difficult to use with an aqueous solution alone. For example, a first aqueous solution of the prebiotic/s and the probiotic/s, and a second solution of the coating material/s in an organic solvent may be used in each nozzle.

Advantageously, the various embodiments described allows for the formulation of relatively high contents of the probiotic, which are sensitive to the spray drying and storage conditions. Advantageously, the use of two different coating materials could provide complementary benefits from different coating materials. Advantageously, the various embodiments described may be used with a variety of food matrices and may be tailored to suit the specific food product. For example, the food product may be beer, ice-cream, an ice block, or milk powder.

### DETAILED DESCRIPTION

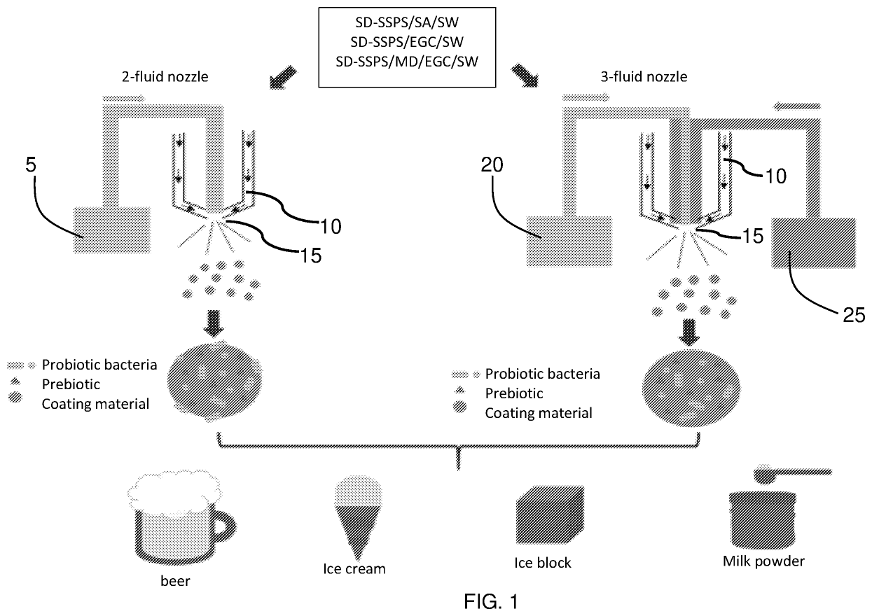

FIG. 1 shows a schematic diagram of a preparation of spray-dried compositions.

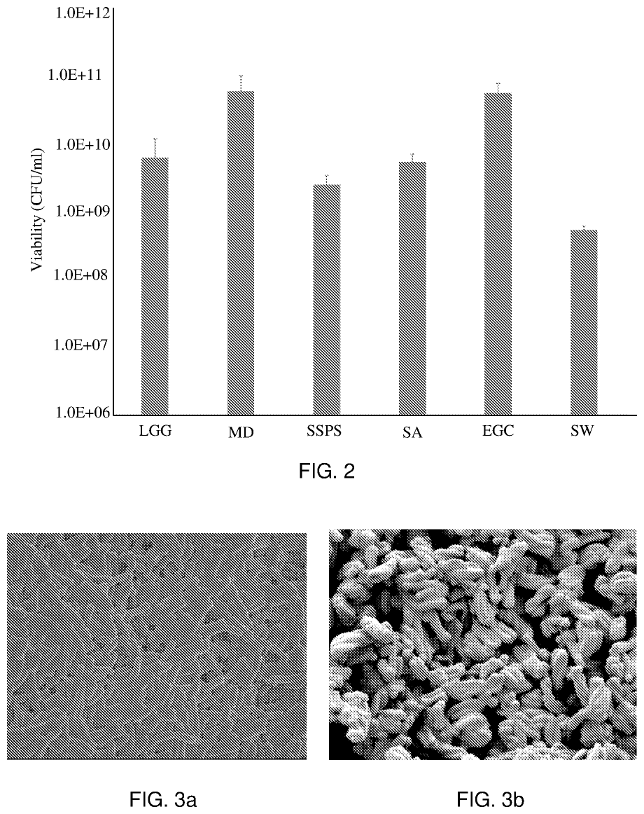

FIG. 2 shows the effect of selected excipients on the viability of probiotic bacteria Lacticaseibacillus rhamnosus GG (LGG) including maltodextrin (MD), soluble soy polysaccharide (SSPS), sodium alginate (SA), poly(ethylacrylate-co-methyl methacrylate) 2:1 (Eudraguard® Control) (EGC), and Swanlac® ASL10 (SW). All the excipients were tested at 5% (w/v), except for SA which was tested at 2.5% (w/v) due to the viscosity of the solution.

FIG. 3 show field emission scanning electron microscope (FESEM) images of the morphology of free LGG and spry-dried LGG (control) in FIG. 3a and FIG. 3b respectively.

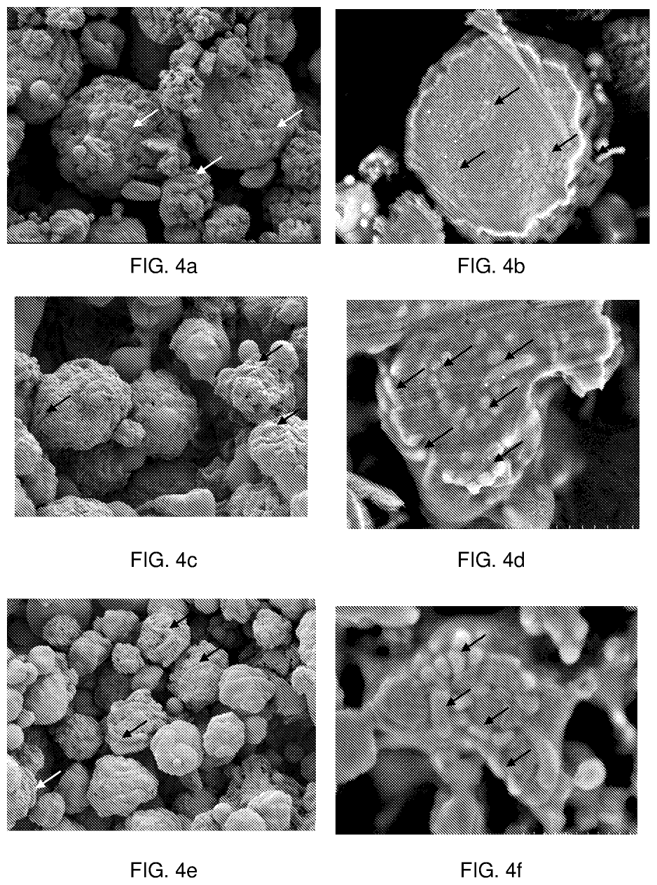

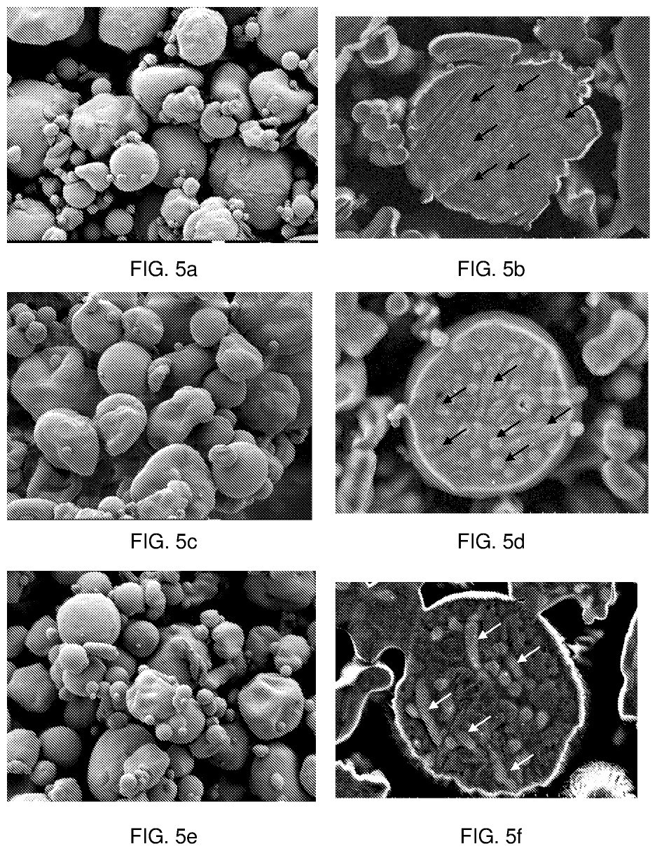

FIG. 4 show FESEM images of the various spray-dried compositions using a two-fluid nozzle (2F). FIGS. 4a and 4b respectively show the morphology and microstructure of the SD-SSPS/SA/SW spray-dried composition. FIGS. 4c and 4d respectively show the morphology and microstructure of the SD-SSPS/EGC/SW sprayed dried composition. FIGS. 4c and 4f respectively show the morphology and microstructure of the SD-SSPS/MD/EGC/SW sprayed dried composition. The arrows in FIGS. 4 and 5 denote representative probiotic bacteria.

FIG. 5 show FESEM images of the various spray-dried compositions using a three-fluid nozzle (3F). FIGS. 5a and 5b respectively show the morphology and microstructure of the SD-SSPS/SA/SW spray-dried composition. FIGS. 5c and 5d respectively show the morphology and microstructure of the SD-SSPS/EGC/SW sprayed dried composition. FIGS. 5e and 5f respectively show the morphology and microstructure of the SD-SSPS/MD/EGC/SW sprayed dried composition.

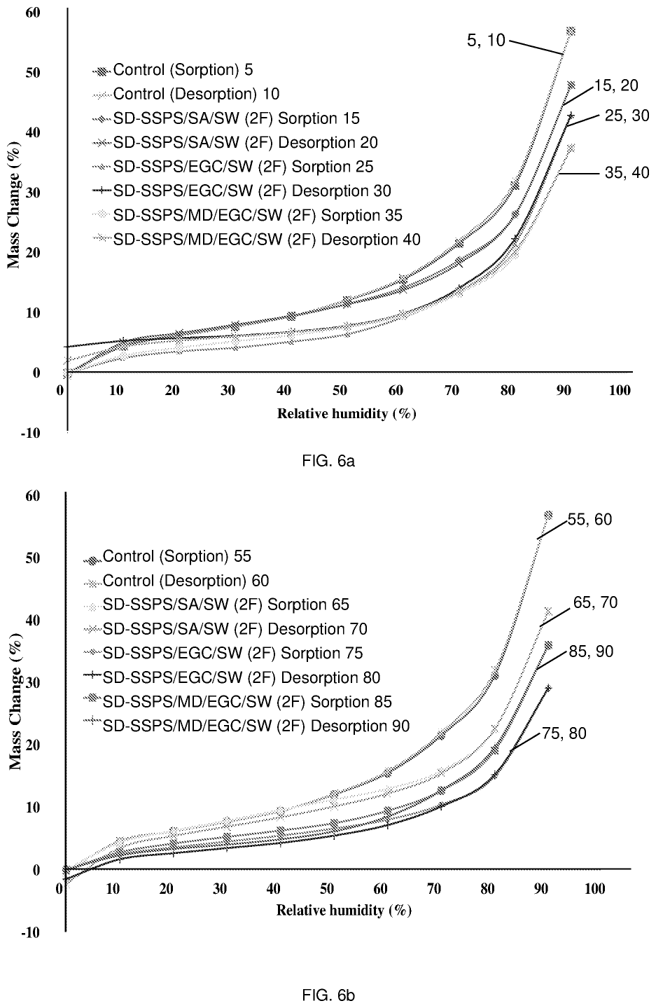

FIGS. 6a and 6b respectively show the dynamic vapour sorption isotherm of control and spray-dried encapsulated probiotic powder formulations prepared using a 2-fluid nozzle and a 3-fluid nozzle.

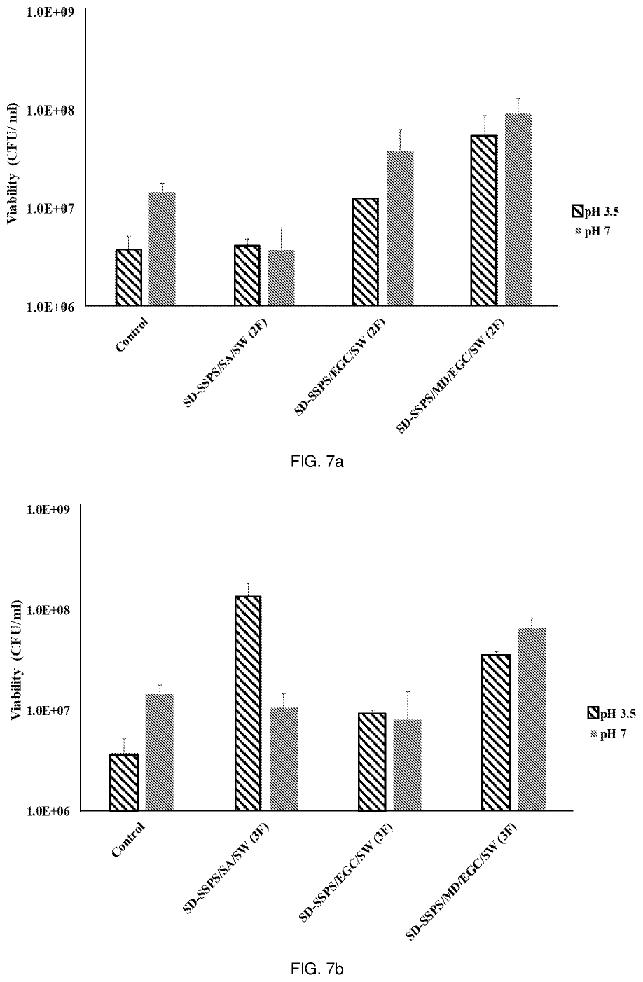

FIGS. 7a and 7b respectively show the viability of control and spray-dried encapsulated LGG prepared with a 2-fluid nozzle and a 3-fluid nozzle after 3 h exposure in pH 3.5 and pH 7. The bar on the left of each sample is for pH 3.5, and the bar on the right is for pH 7.

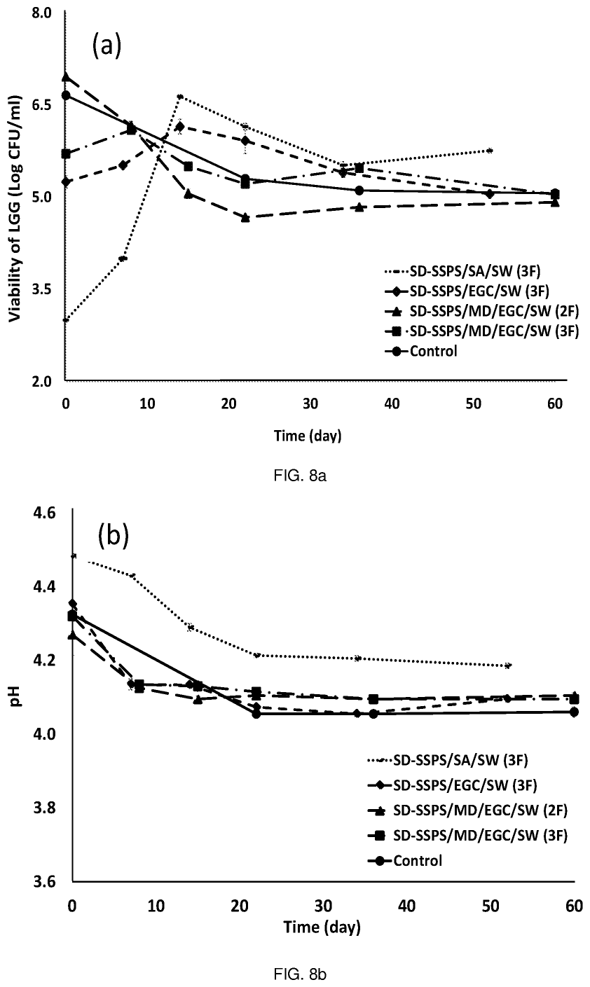

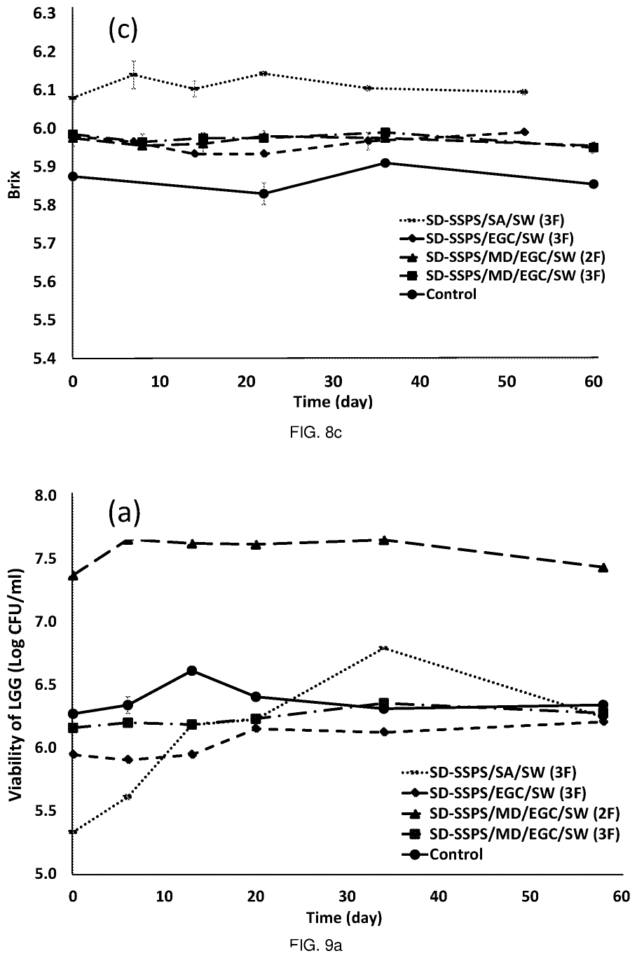

FIGS. 8a, 8b and 8c respectively show the viability of the probiotic in beer over time, changes in pH over time, and changes in Brix of the beer over time.

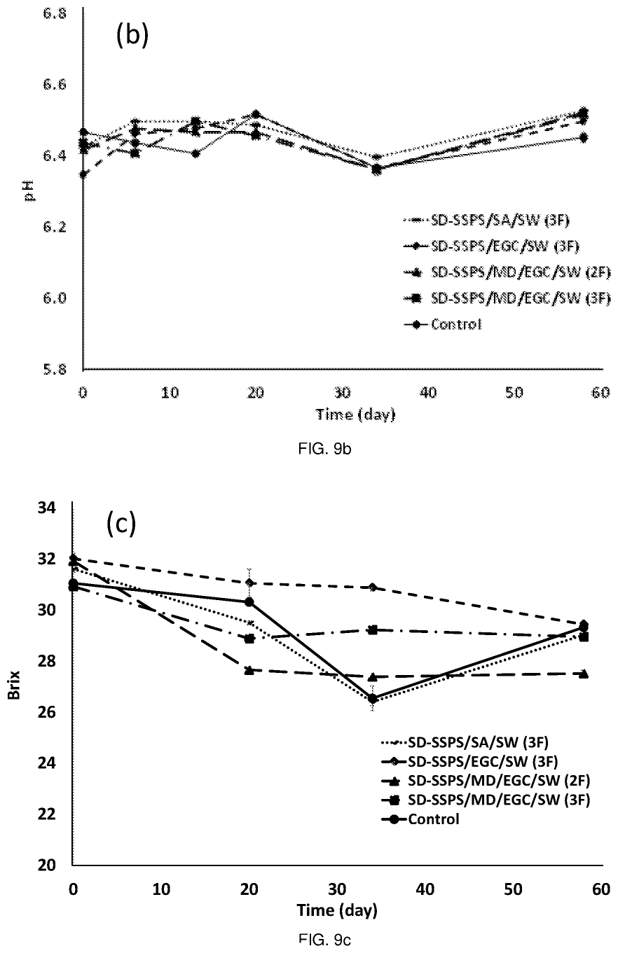

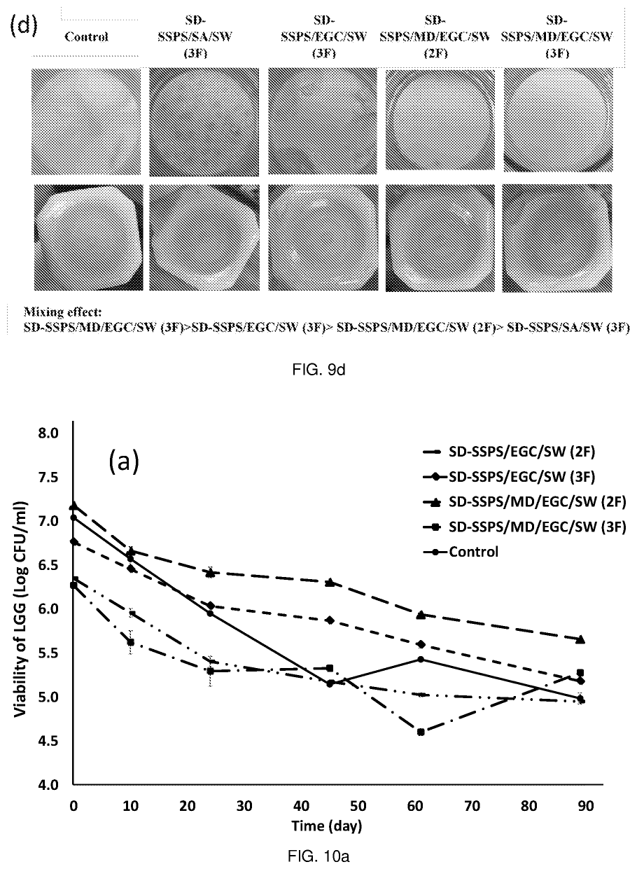

FIGS. 9a, 9b, and 9c respectively show the viability of control and spray-dried encapsulated LGG in ice cream over time, changes in pH over time, and changes in Brix over time. FIG. 9d shows the appearance of ice-cream added with control and various spray-dried encapsulated LGG powders under storage at −20° C.

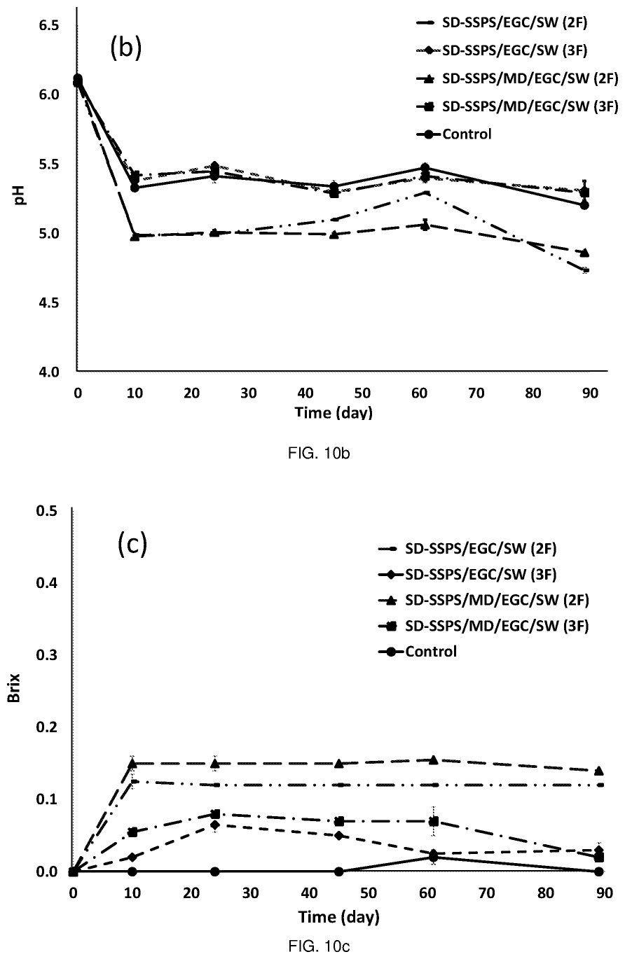

FIGS. 10a, 10b, and 10c respectively show the viability of control and spray-dried encapsulated LGG in an ice block over time, changes in pH over time, and changes in Brix over time. The control and various spray-dried encapsulated LGG powders were added to water, froze, and kept under storage at −20° C.

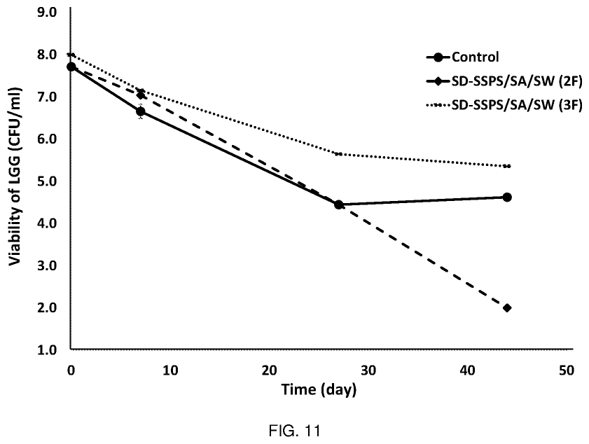

FIG. 11 shows the viability of control and spray-dried encapsulated LGG in milk powder at 30° C.

It will be understood, however, to one skilled in the art, that embodiments of the invention may be practiced without some or all of these specific details. Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of a product and/or apparatus may correspondingly be applicable to a method, even if not explicitly described.

Unless defined otherwise, all technical and scientific terms used herein have the meaning commonly understood by a person skilled in the art to which this invention belongs.

The terms “about”, “approximately”, “substantially” must be read with reference to the context of the application as a whole, and have regard to the meaning a particular technical term qualified by such a word usually has in the field concerned. For example, it may be understood that a certain parameter, function, effect, or result can be performed or obtained within a certain tolerance, and the skilled person in the relevant technical field knows how to obtain the tolerance of such term.

Ranges may be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if a range from 10 to 15 is disclosed, then 10, 11, 12, 13, 14 and 15 are considered disclosed.

The phrase “at least one of A and B” means it requires only A alone, B alone, or A and B, i.e. only one of A or B is required. The phrase “A and/or B” includes A alone, B alone and A and B.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Oligosaccharides generally refer to a few monosaccharides linked together (typically three to 10 monosaccharides, but may be more and up to 39 monosaccharides), and may be linked to lipids or amino acid side chains. Polysaccharides generally refer to long chain polymeric monosaccharide units, may be linear or branched, and may contain one or more monosaccharide. Polysaccharides typically have 40 to 3000 monosaccharides.

Shellac may be provided in the form of a food grade aqueous shellac solution (e.g. Swanlac® ASL 10 which contains aqueous ammonium shellac with a solid content of 25%), or ethanolic shellac solution.

Poly(ethylacrylate-co-methyl methacrylate) 2:1 is a copolymer of ethyl acrylate and methyl methacrylate with an approximate 2:1 ratio of ethyl acrylate to methyl methacrylate and is commercially available as Eudraguard® Control. Other copolymers mentioned herein are similarly named as per convention.

A combined ‘HARDWARE’ (benign spray drying technology) and ‘SOFTWARE’ (matrix-dependent novel formulation; ‘toolbox concept−prebiotic+probiotic+coating material’) approach which may be specifically tailored to a variety of dairy and non-dairy food matrices to achieve a synergistic response is described herein.

Three encapsulated probiotic powder formulations (containing prebiotic/s, probiotic and coating material/s) that could be readily prepared using benign spray drying technique via two-fluid or three-fluid nozzles have been developed based on the above approach. The spray-dried encapsulated probiotic powder formulations could be incorporated into a variety of food matrices (e.g. beer, ice-cream, ice block and milk powder) with enhanced probiotic survival and stability as compared to the non-encapsulated species. These powder formulations would collectively make up a formulation toolbox which could aid the formulator in developing the most robust probiotic formulation for the specific food product. It is envisaged that different kinds of food may require different probiotic formulations in their matrices, and this toolbox will no doubt speed up the search and/or development of the most optimal one. In various embodiments, the probiotic is present in 10 to 80 weight percent (wt. %), the prebiotic is present in 5 to 50 wt. %, and the coating material is present in 30 to 80 wt. %, with a combined total of 100 wt. %. Preferably, the probiotic is present in 10 to 60 wt. %, preferably 10 to 50 wt. %, more preferably 10 to 40 wt. %, and even more preferably 10 to 30 wt. %.

In FIG. 2, it may be observed that among the selected excipients, maltodextrin (MD) and poly(ethylacrylate-co-methyl methacrylate) 2:1 (EGC) improve the viability of Lacticaseibacillus rhamnosus GG (LGG) in the tested conditions. Lacticaseibacillus rhamnosus was previously known as Lactobacillus rhamnosus when a taxonomic revision reassigned many species from Lactobacilli into 25 genera [7]. LGG is a specific strain of Lacticaseibacillus rhamnosus with American Type Culture Collection (ATCC) accession number 53103.

### Spray-Dried Encapsulated Probiotic (LGG) Powder Formulations

Three probiotic powder formulations (SD-SSPS/SA/SW, SD-SSPS/EGC/SW and SD-SSPS/MD/EGC/SW), differing in type and amount of prebiotics (i.e. soluble soy polysaccharides and maltodextrin) and coating materials (sodium alginate, poly(ethylacrylate-co-methyl methacrylate) 2:1 and shellac) were prepared via spray drying. A combination of different coating materials may be used which could provide complementary benefits from the different coating materials. The formulations could be spray-dried using either two-fluid (2F) or three-fluid nozzles (3F) (Table 1, FIG. 4 and FIG. 5) with a conventional spray drying apparatus, which thus showed the formulations' processing versatilities.

In an example of a SD-SSPS/SA/SW sample prepared by a 2-fluid nozzle, an aqueous solution containing 15 to 22 wt. % of Lacticaseibacillus rhamnosus GG, 9 to 11 wt. % of soluble soy polysaccharide, 33 to 35 wt. % of sodium alginate, and 36 to 39 wt. % of shellac (Swanlac® ASL 10, SW) was prepared with the total weight percentage of all the components making up to 100 wt. %. The solution was spray dried using an apparatus with a 2-fluid nozzle. The total concentration of the feed solution is fixed at 2 wt. %, respectively in this example. The following spray drying conditions were used in this example: an inlet temperature of 100° C., an atomization rate of 742 L/h and an aspiration rate of 35 m³/h.

In an example of a SD-SSPS/SA/SW sample prepared by a 3-fluid nozzle, an aqueous solution containing 15 to 22 wt. % of Lacticaseibacillus rhamnosus GG, 9 to 11 wt. % of soluble soy polysaccharide, and 33 to 35 wt. % of sodium alginate was prepared. A solution in an organic solvent with 36 to 39 wt. % of shellac (Swanlac® ASL 10, SW) was prepared. Any suitable organic solvent may be used, non-limiting examples include alcohols (like methanol, ethanol, isopropanol) and acetone. The organic solvent is preferably miscible with water and has a relatively low boiling point. The weight percentages of the components in the aqueous solution and organic solution are provided based on the weight percentage of both solutions combined together. The total concentration of the inner and outer feed solutions are fixed at 2 wt. %, respectively in this example. The following spray drying conditions were used in this example: an inlet temperature of 100° C., an atomization rate of 742 L/h and an aspiration rate of 35 m³/h. The spray-drying apparatus may be equipped with both a two-fluid nozzle and a three-fluid nozzle or may be interchangeable between the two and three fluid nozzles. Alternatively, two separate apparatuses each with two and three fluid nozzles may be used.

TABLE 1
Composition of spray dried encapsulated probiotic powder formulations

| | Amount (wt. %)* | | |
|---|---|---|---|
| | SD- | SD- | SD- |
| | SSPS/SA/SW | SS[S/EGC/SW | SSPS/MD/EGC/SW |
| Ingredient | (2F, 3F) | (2F, 3F) | (2F, 3F) |
| Lacticaseibacillus | 15-22 | 15-22 | 15-22 |
| rhamnosusGG (LGG) | | | |
| soluble soy polysaccharide | 9-11 | 33-35 | 9-11 |
| (SSPS) | | | |
| maltodextrin (MD) | — | — | 33-35 |
| sodium alginate (SA) | 33-35 | — | — |
| poly(ethylacrylate-co-methyl | — | 18-20 | 9-10 |
| methacrylate) 2:1 | | | |
| (Eudraguard ® Control, EGC) | | | |
| Shellac (Swanlac ® ASL10, | 36-39 | 27-30 | 27-29 |
| SW) | | | |

*a range of quantity was used (batch to batch variation)

### Morphology and Microstructure of the Probiotic Bacteria and Spray-Dried Particles Using 2-Fluid and 3-Fluid Nozzles

The spray-dried encapsulated probiotic particles (i.e. SD-SSPS/SA/SW (2F), SD-SSPS/EGC/SW (2F) and SD-SSPS/MD/EGC/SW (2F)) prepared using the 2-fluid nozzle had a rough surface with corrugated morphology (see FIGS. 4a, 4c, and 4e respectively). A small amount of probiotic bacteria was found to be residing on the surface of the particles. Microstructural analysis show that the particles had a solid interior with most of the probiotic bacteria being encapsulated within the particles.

The spray-dried encapsulated probiotic particles (i.e. SD-SSPS/SA/SW (3F), SD-SSPS/EGC/SW (3F) and SD-SSPS/MD/EGC/SW (3F)) prepared using the 3-fluid nozzle had a mixed smooth spherical and dimpled surface morphology (see FIGS. 5a, 5c, and 5e respectively). Microstructural analysis show that the particles had a solid interior with the probiotic cells being encapsulated within the particles. Unlike particles prepared using the 2-fluid nozzle, exposure of probiotic bacteria on the surface of the particles was not observed (FIG. 4a-c (left)).

### Water Activity, Moisture Content, and Hygroscopicity of Spray-Dried Probiotic Powders

TABLE 2
Water activity, moisture content and hygroscopicity
of spray-dried probiotic powders

| | | | Hygroscopicity |
|---|---|---|---|
| | Water | Moisture | (gram water/ |
| Formulation | activity, a_{w} | content (%) | gram powder |
| 2F | | | |
| SD-SSPS/SA/SW | 0.53 ± 0.01 | 10.60 ± 0.10 | 9.95 ± 1.28 |
| SD-SSPS/EGC/SW | 0.43 ± 0.01 | 3.76 ± 0.65 | 3.11 ± 2.27 |
| SD-SSPS/MD/EGC/SW | 0.42 ± 0.00 | 6.41 ± 0.82 | 3.24 ± 0.69 |
| 3F | | | |
| SD-SSPS/SA/SW | 0.35 ± 0.02 | 9.42 ± 0.43 | 6.87 ± 1.31 |
| SD-SSPS/EGC/SW | 0.38 ± 0.02 | 3.01 ± 0.06 | 3.59 ± 0.16 |
| SD-SSPS/MD/EGC/SW | 0.34 ± 0.00 | 5.44 ± 0.21 | 2.39 ± 0.56 |

Data are presented as average ± standard deviation

Table 2 shows the water activity, moisture content and hygroscopicity of spray-dried encapsulated probiotic powders. The spray-dried encapsulated probiotic powders had a water activity in the range of 0.34-0.53, which was below the threshold (a_{w}=0.6) for the growth of most microorganisms [8]. The more hygroscopic a formulation is, the higher the tendency of the formulation to absorb moisture. Formulations with higher hygroscopicity were found to have higher moisture content (i.e. SD-SSPS/SA/SW (2F, 3F)). In general, the formulations produced via the 3-fluid nozzle were more moisture-stable compared to the corresponding 2-fluid nozzle-produced counterparts.

### Dynamic Vapour Sorption (DVS)

FIG. 6 show the dynamic vapour sorption (DVS) isotherm of control and spray-dried encapsulated probiotic powder formulations. The DVS isotherm for the formulations prepared using a 2-fluid nozzle is shown in FIG. 6a and in FIG. 6b for the formulations prepared using a 3-fluid nozzle. All the formulated powders were found to be less susceptible to moisture uptake than the control and had lower maximum mass change when compared to the control (FIGS. 6a and 6b). In general, the formulations produced via the 3-fluid nozzle were more moisture-stable compared to their corresponding 2-fluid nozzle-produced counterparts.

In both FIG. 6a and FIG. 6b, the sorption and desorption lines for each formulation generally overlap especially as the relative humidity increases and are labelled together. At low relative humidity levels, the percentage change in mass is generally similar for all the samples. In FIG. 6a, the sorption and desorption lines 5, 10 of the control sample show an increased percentage change in mass compared to the two-fluid (2F) nozzle prepared formulations from about 50% relative humidity. At 90% relative humidity, the percentage change in mass decreases in the following order the SD-SSPS/SA/SW formulation 15, 20, the SD-SSPS/EGC/SW formulation 25, 30, and the SD-SSPS/MD/EGC/SW formulation 35, 40. For the latter two, their percentage change in mass starts to deviate from about 80% relative humidity. FIG. 6b shows a similar trend for the samples prepared by the three-fluid (3F) nozzle with the control sample 55, 60 having the highest percentage change in mass. The SD-SSPS/SA/SW formulation 65, 70 is the next highest. However, the order is switched for the SD-SSPS/EGC/SW formulation 75, 80, and the SD-SSPS/MD/EGC/SW formulation 85, 90 with the former formulation having the lowest percentage change in mass when prepared by the three-fluid nozzle. It may also be observed that the difference in the percentage change in mass between the control and the formulated samples is significantly larger for the samples prepared by the three-fluid nozzle (FIG. 6b) indicating improved moisture stability compared to the samples prepared by the two-fluid nozzle (FIG. 6a).

### Viability of Spray-Dried Encapsulated LGG

FIG. 7 shows the viability of the spray-dried encapsulated LGG probiotic bacteria at different pH values. There was a significant reduction of survivability of non-encapsulated LGG (control, unprotected) at low pH (of 3.5) as compared to that at neutral pH (of 7). On the other hand, most spray-dried encapsulated LGG (except SD-SSPS/EGC/SW (2F) and SD-SSPS/SA/SW (3F)) (FIGS. 7a and 7b) show improved performance (i.e. narrowed margin of difference between the species in pH 3.5 and pH 7). This protective effect was even more prominent in spray-dried encapsulated LGG prepared using a 3-fluid nozzle (FIG. 7b).

Incorporation of Spray-Dried Encapsulated Probiotic Powder into Various Food Matrices

FIG. 8a shows the viability of a control sample and various spray-dried encapsulated LGG formulations in unhopped beer under storage at 30° C. for 60 days. FIG. 8b shows the changes in pH and FIG. 8c shows the changes in Brix of the beer. The survival of the probiotic LGG in spray-dried encapsulated forms was much higher than the control (FIG. 8a) (when each of them was compared to its viability at t=0), except for SD-SSPS/MD/EGC/SW (2F), after the beer was stored at 30° C. for approximately two months. The changes in the pH and Brix content of the beer with the addition of the spray-dried encapsulated LGG powder formulations were negligible (see FIGS. 8b and 8c), and that the formulations do not adversely affect the beer.

FIG. 9a shows the viability of a control sample and various spray-dried encapsulated LGG formulations in ice cream (soft serve ice cream from McDonald's was used) under storage at −20° C. for 60 days. FIG. 9b shows the changes in pH and FIG. 9c shows the changes in Brix of the ice cream. FIG. 9d shows the appearance of the ice cream with the control and powders under storage at −20° C.

The survival of LGG in spray-dried encapsulated forms was higher than the control (FIG. 9a) (when each of them was compared to its viability at t=0), except for SD-SSPS/MD/EGC/SW (2F) and SD-SSPS/MD/EGC/SW (3F), after the ice cream was stored at −20° C. for approximately two months. The changes in pH and Brix content of the ice cream with the addition of spray-dried encapsulated LGG powder were negligible. However, there was a significant drop in Brix content for the ice cream added with SD-SSPS/SA/SW (3F) and control (FIG. 9c) after storage −20° C. for approximately one month, which subsequently increased again. Both SD-SSPS/EGC/SW (3F) and SD-SSPS/MD/EGC/SW (3F) had a good mixing effect with ice cream (see FIG. 9d) without any visible changes to the ice cream.

FIG. 10a shows the viability of a control sample and spray-dried encapsulated LGG formulations in an ice block under storage at −20° C. for 60 days. FIG. 10b shows the changes in pH and FIG. 10c shows the changes in Brix of an ice block added with control and various spray-dried encapsulated LGG powders.

The survival of LGG in spray-dried encapsulated forms was much higher than the control (FIG. 10a) (when each of them was compared to its viability at t=0), after the ice block was stored at −20° C. for approximately three months. The changes in pH and Brix content of the ice block with the addition of SD-SSPS/EGC/SW (3F) and SD-SSPS/MD/EGC/SW (3F) were negligible (see FIGS. 10b and 10c).

FIG. 11 shows the viability of a control sample and spray-dried encapsulated LGG formulations in milk powder stored at 30° C. for approximately one and a half months. The survival of LGG in SD-SSPS/SA/SW (3F) was higher than the control (FIG. 11) (when each of them was compared to its viability at t=0).

The formulations may be incorporated into the various food matrices by any means. For example, the spray-dried compositions may be added to a liquid like beer and stored. The spray-dried compositions may also be added to water or soft serve ice cream at room temperature and subsequently freezing the food product with the composition. The spray-dried composition may also be added directly and mixed with a solid food matrix like milk powder.

### CONCLUSION

The formulation toolbox may be applied to other food matrices and the various formulations have their unique applications in different food matrices. In the examples described herein, SD-SSPS/SA/SW (3F), SD-SSPS/EGC/SW (3F) and SD-SPS/MD/EGC/SW (3F) appear to be the most versatile formulations and could be applied across most of the different food categories.

Whilst there has been described in the foregoing description preferred embodiments of the invention, it will be understood by those skilled in the field concerned that many variations or modifications in details of design or construction may be made without departing from the present invention.

### REFERENCES

[1] R. I. Corona-Hernandez, E. Alvarez-Parrilla, J. Lizardi-Mendoza, A. R. Islas-Rubio, L. A. de la Rosa, A. Wall-Medrano, Structural stability and viability of microencapsulated probiotic bacteria: a review, Comprehensive Reviews in Food Science and Food Safety, 12 (2013) 614-628.
[2] International Dairy Federation, Guideline for the enumeration of Bifidobacteria in fermented dairy products, 340 (1999) 19-23.
[3] A. M. Mortazavian, S. H. Razavi, M. R. Ehsani, S. Sohrabvandi, Principles and methods of microencapsulation of probiotic microorganisms, Iranian Journal of Biotechnology, 5 (2007) 1-17.
[4] B. Arribas, N. Garrido-Mesa, L. Peran, D. Camuesco, M. Comalada, E. Bailon, M. Olivares, J. Xaus, L. Kruidenier, I. R. Sanderson, A. Zarzuelo, M. E. Rodriguez-Cabezas, J. Galvez, The immunomodulatory properties of viable Lactobacillus salivarius ssp. salivarius CECT5713 are not restricted to the large intestine, European Journal of Nutrition, 51 (2012) 365-374.
[5] G. Broeckx, D. Vandenheuvel, I. J. J. Claes, S. Lebeer, F. Kiekens, Drying techniques of probiotic bacteria as an important step towards the development of novel pharmabiotics, International Journal of Pharmaceutics, 505 (2016) 303-318.
[6] Market Research.com, Microencapsulation to Boost Innovation in Food & Beverage Industry (https://blog.marketresearch.com/microencapsulation-to-boost-innovation-in-food-beverage-industry), 2017.
[7] J. Zheng, S. Wittouck, E. Salvetti, C. M. A. P. Franz, H. M. B. Harris, P. Mattarelli, P. W. O'Toole, B. Pot, P. Vandamme, J. Walter, K. Watanabe, A taxonomic note on the genus Lactobacillus: Description of 23 novel genera, emended description of the genus Lactobacillus Beijerinck 1901, and union of Lactobacillus and Leuconostocaceae. International Journal of Systematic and Evolutionary Microbiology. 70 (4) (2020) 2782-2858.
[8] Tapia, M., S. Alzamora, and J. Chirife, Effects of Water Activity (aw) on Microbial Stability: As a Hurdle in Food Preservation, 2008. p. 239-271.

1. A spray-dried composition comprising 5 to 50 weight percent (wt. %) of a prebiotic, 10 to 65 wt. % of a probiotic, and 30 to 80 wt. % of a coating material,
wherein the prebiotic is selected from the group consisting of a polysaccharide, an oligosaccharide, a polyol, whey protein, and any combinations thereof;
wherein the probiotic is from a genus selected from the group consisting of Lacticaseibacillus, Lactobacillus, Lactiplantibacillus, Levilactobacillus, Ligilactobacillus, Limosilactobacillus, Bifidobacterium, Enterococcus, Streptococcus, Pediococcus, Leuconostoc, Bacillus, Escherichia, Saccharomyces, and any combinations thereof;
wherein the coating material is selected from the group consisting of a polymer of acrylic acid and/or acrylates, shellac, alginic acid and its salts, and any combinations thereof.

2. The spray-dried composition according to claim 1, wherein the probiotic is present in 10 to 60 wt. %, preferably 10 to 50 wt. %, more preferably 10 to 40 wt. %, and even more preferably 10 to 30 wt. %.

3. The spray-dried composition according to claim 1, wherein the prebiotic is selected from the group consisting of soluble soy polysaccharides, maltodextrin, inulin, oligofructose, fructo-oligosaccharides, galacto-oligosaccharides, pectic oligosaccharides, xylooligosaccharides, soya bean oligosaccharides, isomaltooligosaccharides, glucooligosaccharides, arabinoxyloligosaccharides, raffinose, palatinose, lactosucrose, resistant starch and polydextrose, plant-derived and fungal-derived polysaccharides, mannitol, xylitol, sorbitol, lactulose, lactitol, maltitol, trehalose, lactose, whey protein isolate, whey protein concentrate, and any combinations thereof.

4. The spray-dried composition according to claim 3, wherein the prebiotic is the polysaccharide, preferably the prebiotic comprises soluble soy polysaccharides and/or maltodextrin.

5. The spray-dried composition according to claim 1, wherein the genus of the probiotic is Lacticaseibacillus or Lactobacillus.

6. The spray-dried composition according to claim 5, wherein the probiotic is Lacticaseibacillus rhamnosus.

7.-8. (canceled)

9. The spray-dried composition according to claim 1, wherein the polymer of acrylic acid and/or acrylates is selected from the group consisting of poly(ethylacrylate-co-methyl methacrylate), poly(methacrylic acid-co-methyl methacrylate), poly(methacrylic acid-co-ethyl acrylate), poly(methyl acrylate-co-methyl methacrylate-co-methacrylic acid), and any combinations thereof.

10. The spray-dried composition according to claim 9, wherein the polymer of acrylic acid and/or acrylates is selected from the group consisting of poly(ethylacrylate-co-methyl methacrylate) with an approximate ratio of 2:1 to 1:1 of ethyl acrylate to methyl methacrylate, poly(methacrylic acid-co-methyl methacrylate) with an approximate ratio of 1:1 to 1:2 of methacrylic acid to methyl methacrylate, poly(methacrylic acid-co-ethyl acrylate) with an approximate ratio of 1:1 of methacrylic acid to ethyl acrylate, poly(methyl acrylate-co-methyl methacrylate-co-methacrylic acid) with an approximate ratio of 7:3:1 of methyl acrylate to methyl methacrylate to methacrylic acid, and any combinations thereof.

11. The spray-dried composition according to claim 1, wherein there are at least two different polymers.

12. The spray-dried composition according to claim 1, wherein the prebiotic is distinct or different from the coating material.

13. The spray-dried composition according to claim 12 with one of the following compositions:
(i) Lacticaseibacillus rhamnosus GG is present in 15 to 22 wt. %, soluble soy polysaccharides are present in 9-11 wt. %, sodium alginate is present in 33-35 wt. %, and shellac is present in 36-39 wt. %;
(ii) Lacticaseibacillus rhamnosus GG is present in 15 to 22 wt. %, soluble soy polysaccharides are present in 33-35 wt. %, poly(ethylacrylate-co-methyl methacrylate) 2:1 is present in 18-20 wt. %, and shellac is present in 27-30 wt. %; and
(iii) Lacticaseibacillus rhamnosus GG is present in 15 to 22 wt. %, soluble soy polysaccharides are present in 9-11 wt. %, maltodextrin is present in 33-35 wt. %, poly(ethylacrylate-co-methyl methacrylate) 2:1 is present in 9-10 wt. %, and shellac is present in 27-29 wt. %.

14. The spray-dried composition according to claim 1, wherein at least one of the following conditions are fulfilled:
(i) a majority of the particles in the spray-dried composition has a rough surface;
(ii) the spray-dried composition is prepared by a two-fluid nozzle;
(iii) wherein a majority of the particles in the spray-dried composition has a smooth and dimpled surface; and
(iv) the spray-dried composition is prepared by a three-fluid nozzle.

15. The spray-dried composition according to claim 1 with a water activity of less than 0.6, preferably 0.55 or less.

16. A method of preparing a spray-dried composition, the method comprising:
providing at least one solution comprising 5 to 50 weight percent (wt. %) of a prebiotic, 10 to 65 wt. % of a probiotic, and 30 to 80 wt. % of a coating material,
wherein the prebiotic is selected from the group consisting of oligosaccharides, polysaccharides, polyols, whey protein and any combinations thereof,
wherein the probiotic is from a genus selected from the group consisting of Lacticaseibacillus, Lactobacillus, Lactiplantibacillus, Levilactobacillus, Ligilactobacillus, Limosilactobacillus, Bifidobacterium, Enterococcus, Streptococcus, Pediococcus, Leuconostoc, Bacillus, Escherichia, Saccharomyces, and any combinations thereof;
wherein the coating material is selected from the group consisting of a polymer of acrylic acid and/or acrylates, shellac, alginic acid and its salts, and any combinations thereof; and
spray drying the at least one solution to obtain the spray-dried composition.

17. The method according to claim 16, wherein the probiotic is present in 10 to 60 wt. %, preferably 10 to 50 wt. %, more preferably 10 to 40 wt. %, and even more preferably 10 to 30 wt. %.

18. The method according to claim 16, wherein at least one of the following conditions is fulfilled:
(a) the prebiotic is selected from the group consisting of soluble soy polysaccharides, maltodextrin, inulin, oligofructose, fructo-oligosaccharides, galacto-oligosaccharides, pectic oligosaccharides, xylooligosaccharides, soya bean oligosaccharides, isomaltooligosaccharides, glucooligosaccharides, arabinoxyloligosaccharides, raffinose, palatinose, lactosucrose, resistant starch, polydextrose, plant-derived and fungal-derived polysaccharides, mannitol, xylitol, sorbitol, lactulose, lactitol, maltitol, trehalose, lactose, whey protein isolate, whey protein concentrate, and any combinations thereof;
(b) the prebiotic is the polysaccharide, preferably soluble soy polysaccharides and/or maltodextrin;
(c) the genus of the probiotic is Lacticaseibacillus or Lactobacillus, preferably the probiotic is Lacticaseibacillus rhamnosus;
(d) the synthetic polymer and/or the natural polymer of the coating material each have a carboxylic acid functional group or an ester functional group;
(e) there are at least two different polymers; and
(f) the prebiotic is distinct or different from the coating material.

19. The method according to claim 18, wherein the polymer of acrylic acid and/or acrylates is selected from the group consisting of poly(ethylacrylate-co-methyl methacrylate), poly(methacrylic acid-co-methyl methacrylate), poly(methacrylic acid-co-ethyl acrylate), poly(methyl acrylate-co-methyl methacrylate-co-methacrylic acid), and any combinations thereof.

20. The method according to claim 19, wherein the polymer of acrylic acid and/or acrylates is selected from the group consisting of poly(ethylacrylate-co-methyl methacrylate) with an approximate ratio of 2:1 to 1:1 of ethyl acrylate to methyl methacrylate, poly(methacrylic acid-co-methyl methacrylate) with an approximate ratio of 1:1 to 1:2 of methacrylic acid to methyl methacrylate, poly(methacrylic acid-co-ethyl acrylate) with an approximate ratio of 1:1 of methacrylic acid to ethyl acrylate, poly(methyl acrylate-co-methyl methacrylate-co-methacrylic acid) with an approximate ratio of 7:3:1 of methyl acrylate to methyl methacrylate to methacrylic acid, and any combinations thereof.

21. The method according to claim 16, wherein spray drying the at least one solution is performed with a two fluid nozzle, preferably, the at least one solution comprises an aqueous solution or spray drying the at least one solution is performed with a three fluid nozzle, preferably the at least one solution comprises an aqueous solution and a solution with an organic solvent.

22. (canceled)

23. A food product comprising the spray-dried composition according to claim 1.
